(19) United States
(12) Patent Application Publication
(10) Pub. No.: US 2002/0187464 A1
(43) Pub. Date: Dec. 12, 2002

(54) Microarray-based method for rapid identification of cells, microorganisms, or protein mixtures

(76) Inventors: Mark S. Klempner, Wayland, MA (US); Jane W. Pepper, North Andover, MA (US); Brian T. Cunningham, Lexington, MA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/957,775

(22) Filed: Sep. 21, 2001

Related U.S. Application Data
(60) Provisional application No. 60/261,440, filed on Jan. 12, 2001.
(60) Provisional application No. 60/234,534, filed on Sep. 22, 2000.

(51) Int. Cl. C12Q 1/70; C12M 1/34; G06F 19/00; G01N 33/48; G01N 33/50

(52) U.S. Cl. 435/5; 435/287.2; 702/19

(57) ABSTRACT

The invention provides compositions and methods for the detection, identification, and quantification of microorganisms, cells, or protein mixtures in a sample.

### PRIORITY

This application claims the benefit of U.S. Provisional Application Ser. No. 60/261,440, filed Jan. 12, 2001, and U.S. Provisional Application Ser. No. 60/234,534, filed Sep. 22, 2000.

### BACKGROUND OF THE INVENTION

In the fields of molecular biology research, pharmaceutical development, high throughput screening, genetics, and diagnostics, many fundamental processes are understood by analyzing the relative chemical binding affinity between different molecules, and by detecting the presence/absence of a chemical within a solution using different types of assays.

One such method for implementing parallel chemical affinity analysis is to place chemical reagents onto a planar solid support, such that the solid support contains many individual locations, each with a different chemical reagent. Such an activated planar solid support is called a “microarray” because the different chemical reagents are typically laid out in a regular grid pattern in x-y coordinates. One possible implementation of a microarray is a DNA microarray, in which each individual location within the array contains a different sequence of oligonucleotides. In this embodiment, the spots within the DNA microarray detect complementary chemical binding with an opposing strand of DNA in a test sample. In order to detect the presence of the opposing DNA when it binds, the opposing DNA is “tagged” with a fluorophor that can be detected by the light emitted when the microarray location is excited with a laser. The original DNA that is placed onto the microarray has high affinity for its complementary DNA sequence, and low affinity for all other DNA sequences.

While the DNA microarray is used to detect and sequence the DNA components of a test sample, a protein microarray can be used to detect the affinity interaction between proteins that are placed onto the individual microarray locations and proteins within a test solution. For example, by placing individual protein antibodies onto different locations on a microarray surface, it is possible to detect the corresponding antigens in a test sample when they bind selectively to the protein antibodies. Like the DNA microarrays, the detected protein can be labeled with a fluorophor to enable detection. Alternatively, some other form of molecular or particle tag can be bound to the detected protein to signal its presence on the microarray surface.

Additional rapid and highly sensitive methods of simultaneous detection of many different chemical affinity interactions between proteins are desirable in the art. In particular methods that do not rely on chemical protein synthesis are needed.

The microarray methodology described in this invention is directly applicable to the detection and identification of complex biological constructs such as cells, viruses, microorganisms, and fungi. Currently available methods for the detection and identification of, for example, bacteria in environmental and biological specimens involve the traditional methods of direct staining for visualization of bacteria, serologic techniques that detect antigens from the bacteria or evidence of prior infection by an antibody response to bacterial antigens, a wide array of culture techniques, and the most recent addition of nucleic acid based molecular techniques (e.g., polymerase chain reaction based amplification methods).

There are numerous stains that are used to visualize and characterize microorganisms. Bacteria are generally visualized with Gram's stain, acid fast stains, modified acid fast stains and fluorescent stains, and fluorescein tagged antibodies that are directed against specific bacteria. Except for direct and indirect fluorescent antibody test, which require a fluorescent microscope and a trained reader of the stained specimen, all of these staining methods depend on the presence of a large number of organisms in the specimen and are not specific enough to identify the species of the bacteria. For example, in a urine sample it requires about 100,000 bacteria/ml to detect greater than 5 bacteria per visual field at a magnification of 400× and staining can provide only an indication of whether the organism is Gram positive or Gram negative. The use of bacterial antigen detection tests is limited to a few species. For example, there are bacterial antigen detection tests that are based on agglutination and are generally used only for cerebrospinal fluid samples to detect Streptococcus pneumoniae, Hemophilus infleunzae type b, Neisseria meningitidis, and Group B streptococci in neonates. Enzyme linked tests are available for detecting Group A streptococci in throat swabs and Legionella pneumophila serotype 1 in sputum and urine specimens. Other antigen agglutination and enzyme immunoassays are available for some fungi, viruses, and parasites.

Identification and quantification of bacteria by in vitro culture and biochemical methods are the “gold standard.” Numerous agar and broth based media are used to cultivate bacteria depending on the source of the specimen and the spectrum of bacteria that are anticipated. Once the bacteria are gown, individual colonies are amplified to obtain pure cultures and subjected to multiple biochemical test and reaction for species identification. While many of these latter steps are currently automated, this technique requires 3 to 5 days to provide information on whether a bacterial infection was present. Specific information on the identity of the microorganism is not available, and many species that reproduce very slowly in culture are not detectable.

Molecular based techniques are of growing importance to diagnostic microbiology. They can be used for organism detection and identification, antimicrobial drug resistance testing and new organism detection. Once organisms have been gown in culture, specific identification can be done using DNA probes that hybridize with bacterial ribosomal RNA. The currently available DNA probes are labeled such that hybridized probes are detected by chemiluminenscence. This method is being used for the identification of only a few pathogens (e.g., sexually transmitted pathogens such as N. gonorrhoeae and C. trachomatis) as well as confirmation of species identification for acid fast bacilli. Polymerase chain reaction (PCR) amplification techniques to detect and identify microorganisms have been under development in many university research laboratories for over 10 years but have been slow to move to the routine diagnostic microbiology laboratory.

The exquisite sensitivity of PCR for detecting very few bacteria is both its major advantage and its greatest drawback. While the identification of a very small number of organisms is very helpful in some specimens (e.g., CSF) or for some bacteria (e.g., M. tuberculosis) in many biologic specimens (e.g., urine, sputum, water, and air) the number of organisms is critical to judge the significance of the finding. Quantitative PCR has been extremely useful of monitoring HIV infection, but standards for bacterial quantification are lacking. Similarly, species specific primers have been designed and validated by the large number of such primers that need to be available for diagnostic microbiology and the problems with false positive reaction, contamination of equipment, and inhibitors of the PCR reaction in biological specimens all present challenges for routine use of this method in the diagnostic microbiology laboratory. Because primers for antimicrobial drug resistance genes have been identified one of the most promising uses of PCR will be in the prediction of antimicrobial agent sensitivity testing. PCR has provided a major advance in our ability to detect previously unidentified species, such as Whipple's disease.

### SUMMARY OF THE INVENTION

It is an object of the invention to provide methods for detecting and identifying receptors and biological constructs. This and other objects of the invention are provided by one or more of the embodiments described below.

One embodiment of the invention provides a solid support comprising at least about ten types of phages, wherein each phage displays a different affinity ligand reagent (ALR), and wherein each type of phage is immobilized to the solid support in a predefined location. Optionally, there can be at least about 100 types of phages or at least about 1,000 types of phages. The ALR can be selected from the group consisting of a polyclonal antibody, a monoclonal antibody, a single chain antibody (scFv), a Fab fragment, a F(ab′)₂ fragment, a Fv fragment, a polypeptide, and a small organic molecule.

Another embodiment of the invention provides a method of detecting at least one receptor in a sample. The method comprises applying the sample to a solid support comprising at least about two types of phages wherein each phage displays a different affinity ligand reagent (ALR), wherein each type of phage is immobilized to the solid support in a predefined location; and detecting the at least one receptor bound to the solid support. Optionally the phages on the solid support that bind the at least one receptor can be identified. The solid support can be washed before detection of the at least one receptor.

The receptor can be selected from the group consisting of a polyclonal antibody, a monoclonal antibody, a single chain antibody (scFv), a Fab fragment, a F(ab′)₂ fragment, a Fv fragment, antisera, an agonist for a cell membrane receptor, an antagonist for a cell membrane receptor, a toxin, a hormone, a hormone receptor, an enzyme, an enzyme substrate, a cofactor, a drug, a lectin, a sugar, an oligonucleotide, a polypeptide, a small organic molecule, a prokaryotic cell, a eukaryotic cell, a cell membrane, a cell membrane receptor, an organelle, and an oligosaccharide. The receptor can also be an outer coat polypeptide of a microorganism selected from the group consisting of bacteria, viruses, fungi, protozoa, and parasites.

The solid support can comprise at least about 100 types of phages each displaying a different ALR. The at least one receptor can be labeled before or after application to the solid support. The label can be selected from the group consisting of a colormetric label, a fluorescent label, a biolunmescent label, a chemiluminescent label, a magnetic particle label, a radioactive label, a labeled antibody specific for a receptor, quantum dot, and a fluorescent particle label. Detection of the receptor can be accomplished by a method selected from the group consisting of detecting modification of mass, detecting refractive index, and detecting surface roughness of the solid support.

Still another embodiment of the invention provides a method of preparing an array of affinity ligand reagents (ALRs). The method comprises the steps of (a) incubating a combinatorial library comprising ALRs with a first biological construct, wherein the biological construct is selected from the group consisting of a cell, bacteria, virus, fungus, protozoa, and parasite; (b) separating ALRs that bind to the biological construct; (c) immobilizing multiple copies of each ALR of step (b) to a solid support in a predefined location; and (d) repeating steps (a)-(b) at least once with at least one additional biological construct and immobilizing each specific ALR to a predefined location on the solid support of step (c). Optionally, the ALR of step (b) can be identified. Optionally, the additional step of determining binding characteristics of each ALR of step (b) to at least about 10 different biological constructs can be carried out. The ARLs can be synthesized on the solid support.

The combinatorial library can be selected from the group of libraries comprising polyclonal antibodies, monoclonal antibodies, single chain antibodies (scFv), Fab fragments, F(ab′)₂ fragments, Fv fragments, small organic molecules, and polypeptides. At least about 10 different ALRs specific for each biological construct can be immobilized on the solid support. The solid support can comprise ALRs derived from at least about 10 different biological constructs. The solid support can be selected from the group consisting of glass, nitrocellulose, nylon, silicon wafers, microfabricated sensors, polystyrene, and polyvinyl chloride.

Even another embodiment of the invention provides a method of preparing an array of phages comprising affinity ligand reagents (ALRs). The method comprises (a) incubating a phage combinatorial library comprising ALRs with a biological construct, wherein the biological construct is selected from the group consisting of a cell, bacteria, virus, fungus, protozoa, and parasite; (b) separating phages that bind to the biological construct; (c) amplifying one or more phages of step (b); (d) immobilizing each specific phage of step (c) to a solid support in a predefined location; (e) repeating steps (a)-(c) at least once with at least one additional biological construct and immobilizing each phage to a predefined location on the solid support of step (d); wherein an array of phages comprising ALRs is prepared. Optionally, steps (a) and (b) can be repeated at least one time for each biological construct. Optionally, the additional step of determining binding characteristics of each phage of step (c) to at least about 10 different biological constructs can be performed.

The ALRs can be selected from the group comprising polyclonal antibodies, monoclonal antibodies, single chain antibodies (scFv), Fab fragments, F(ab′)₂ fragments, Fv fragments, small organic molecules, and polypeptides.

At least about 10 different phages specific for each biological construct can be attached to the solid support. The solid support can comprise specific phages derived from at least about 10 different biological constructs. The solid support can be selected from the group consisting of glass, nitrocellulose, nylon, silicon wafers, microfabricated sensors, polystyrene, and polyvinyl chloride.

Another embodiment of the invention provides a method of preparing an array of affinity ligand reagents (ALRs). The method comprises (a) incubating a phage combinatorial library comprising ALRs with a biological construct, wherein the biological construct is selected from the group consisting of a cell, bacteria, virus, fungus, protozoa, and parasite; (b) separating phages that bind to the biological construct; (c) cloning one or more phages of step (b); (d) isolating the ALR portion of each phage of step (c); (e) immobilizing the ALRs of step (c) to a solid support in a predefined location; and (f) repeating steps (a)-(d) at least once with at least one additional biological construct and immobilizing each ALR to a predefined location on the solid support of step (e). Optionally, steps (a) and (b) are repeated at least one time for each biological construct. Optionally, the additional step of determining binding characteristics of each ALR of step (d) to at least about 10 different biological constructs can be performed. At least about 10 different ALRs specific for each biological construct can be immobilized on the solid support. The solid support can comprise ALRs derived from at least about 10 different biological constructs.

Even another embodiment of the invention provides a method of identifying one or more biological constructs in a sample suspected of containing biological constructs. The method comprises (a) applying the sample to a solid support comprising phages comprising ALRs or ALRs, wherein the ALRs are specific for at least two different biological constructs, wherein at least about 10 different ALRs capable of specifically binding to each of the at least two different biological constructs or at least about 10 different phages comprising ALRs capable of specifically binding to each of the at least two different biological constructs are bound to the solid support in predefined locations and (b) detecting biological constructs bound to the ALRs; whereby a biological construct is identified in the sample. Optionally, the step of detecting the amount of one or more biological constructs bound to the ALRs can be performed, whereby a biological construct is quantified in the sample. The solid support can comprise ALRs specific for at least about 10 different biological constructs. Optionally, the step of washing the solid support before detecting the biological constructs can be performed. The biological constructs can be labeled. The label can be selected from the group consisting of colormetric dye, fluorescent dye, chemiluminescent dye, quantum dots, magnetic particle label, and fluorescent particle label. The biological constructs can be detected by a method selected from the group consisting of gravimetric sensing and phase contrast microscopy. The biological construct can be selected from the group consisting of cells, bacteria, viruses, fungi, protozoa, and parasites. The different biological constructs can comprise different strains of the same biological construct. The sample can be selected from the group consisting of a biological sample, a water sample, an environmental sample, and a food sample.

Still another embodiment of the invention provides a method of providing information concerning biological construct ALR binding patterns to a first computer device through a second computer device. The method comprises (a) receiving at least one ALR binding pattern from the first computer device; (b) comparing the at least one ALR binding pattern received from the first computer device to records in an ALR binding pattern information database; and (c) compiling a list of matching biological construct information from the database records matching the at least one ALR binding pattern received from the first computer device. The step of comparing can comprise (a) obtaining a binding pattern from the first computer device; (b) obtaining a binding pattern record from the database; (c) comparing the binding patterns of steps (a) and (b); and (d) repeating step (c) until a match is made. Step (c) can be repeated until all binding pattern records in the database are compared to the binding pattern from the first computer device.

A computer readable medium can have these instructions stored thereon for causing a central processing unit to execute the method. The list can be visually displayed on a display device. A computer readable medium can have stored therein instructions for visually displaying the list.

The second computer device can be selected from the group of devices consisting of a web server, a stand alone computer, and a personal digital assistant. The first computer device and the second computer device can be connected by a network.

Even another embodiment of the invention provides a method of providing information concerning biological construct or receptor ALR binding patterns and biological construct or receptor quantity to a first computer device through a second computer device. The method comprises (a) receiving at least one ALR binding pattern comprising binding intensities from a first computer device; (b) comparing the at least one ALR binding pattern received from a first computer device to records in an ALR binding pattern and binding intensity information database; and (c) compiling a list of records matching the at least one ALR binding pattern comprising binding intensities received from a first computer device. The second computer device can be selected from the group of devices consisting of a web server, a stand alone computer, and a personal digital assistant. The first computer device and the second computer device can be connected by a network. A computer readable medium can have stored therein instructions for causing a central processing unit to execute the method. The list can be visually displayed on a display device. A computer readable medium can have stored therein instructions for visually displaying the list.

The step of comparing can comprise (a) obtaining a binding pattern comprising binding intensities from the first computer device; (b) obtaining a binding pattern record comprising binding intensities from the database; (c) comparing the binding patterns and intensities of steps (a) and (b); and (d) repeating step (c) until a match is made. Step (c) can be repeated until all records of binding patterns comprising binding intensities in the database are compared to the binding pattern comprising binding intensities from the first computer device.

Another embodiment of the invention provides a system allowing users to obtain information on the identity or quantity of biological constructs or receptors in a sample from a directory available via a computer. The system comprises in combination: (a) a second computer device in communication with a first computer device to allow users to enter selection criteria for retrieving records of biological construct or receptor ALR binding patterns and binding intensities; and (b) a database comprising records of biological construct or receptor ALR binding patterns and binding intensities, wherein the second computer device produces a list of matching ALR binding pattern and binding intensity records from the database that match the selection criteria and displays the matching pattern and binding intensity records on the list in an order determined by each matching biological construct's or receptor's similarity to the selection criteria.

The first computer device and the second computer device can be connected by a network. The selection criteria can comprise an ALR binding pattern provided by the first computer device. The selection criteria can comprise an ALR binding intensity provided by the first computer device. The selection criteria can comprise an ALR binding pattern and an ALR binding intensity provided by the first computer device.

The ALR binding pattern and intensity provided by the first computer device can be determined by applying a sample suspected of containing biological constructs or receptors to a solid support comprising ALRs specific for at least one type of biological construct or receptor, wherein at least about 10 different ALRs capable of specifically binding to each type of biological construct or receptor are bound to the solid support in predefined locations.

The network device can be selected from the group of devices consisting of a web server, a stand alone computer, and a personal digital assistant. The system can further comprise a visual display of the list on a display device. A computer readable medium can have stored therein instructions for visually displaying the list.

Unlike culture-based methods that require several days to multiply bacterial concentration to a sufficient concentration for measurement, the compositions and methods described in this disclosure provide a detection result in less than one hour from, for example, a sample containing less that 1,000 colony forming units and provides positive identification of the species present. This rapid recognition capability can be used in hospital-based diagnostic systems to, for example, provide faster diagnosis of bacteremia and in food/pharmaceutical processing where faster diagnosis of microbial infection will reduce the loss of contaminated product. The invention can provide enormous economic benefit to users through reduced diagnostic costs, decreased morbidity and mortality of patients that are treated rapidly with correct anti-microbial therapy, and reduced operating costs associated with more rapid identification of infected product.

Unlike DNA-based methods such as polymerase chain reaction (PCR), and sequencing-by-hybridization, the method does not require primers for specific target analytes, is not affected by PCR-inhibiting agents normally found in biological samples, does not require extensive sample handling, and does not require thermal cycling/temperature control.

The invention is useful in, for example, applications in which rapid detection and identification of pathogens or receptors provide value in terms of reduced mortality/morbidity, reduced medical care cost, or reduced waste of contaminated process material.

### BRIEF DESCRIPTION OF THE DRAWINGS

FIG. 1 shows a block diagram illustrating an exemplary experimental data processing system for one exemplary embodiment of the present invention

FIG. 2 is a flow diagram illustrating a method for analyzing ALR binding pattern and ALR binding intensity data.

FIG. 3 demonstrates the binding of microorganisms to an array of phages displaying ALRs.

FIG. 4 demonstrates a dose-response curve of one ALR within a phage microarray showing its ability to discriminate between two different microorganisms.

### DETAILED DESCRIPTION OF THE INVENTION

In one embodiment of the invention, phages are affixed to a solid support (e.g., a microarray) so that each individual predefined location of the solid support contains phages that display only one type of differentially displayed affinity ligand reagent (ALR). By placing phage clones onto individual predefined locations on the solid support, it is possible to measure and compare the interaction of many phage clones in parallel with the contents of a test sample. The phages therefore comprise ALRs that are used to query the chemical binding affinities of molecules in a test sample. The microarray format enables detection of the phage-sample interaction by several possible methods.

A phage particle that expresses an ALR on several of its outer surface sites is immobilized on a solid support. The phage-expressed ALR is encoded by a defined region of DNA within the phage. Therefore, the phage effectively is a “particle” that is derivatized with a particular ALR on its outer surface, and can be used directly on a solid support, such as a microarray, to detect the differential chemical binding affinity between the phage-expressed ALR molecules and molecules in a test sample.

Having phages expressing ALRs immobilized to a solid support is desirable because ALRs are always expressed in the same orientation when attached to the phage. Phages also provide an efficient means to produce large quantities of ALRs since their numbers can be amplified by infecting a colony of bacteria. Furthermore, this method is less expensive than protein synthesis, which requires knowledge of the protein amino acid sequence. Additionally, the DNA sequence that encodes for the ALR is automatically carried with every phage and can be used to compare expressed protein or peptide sequences, or to provide a means for protein production with a protein synthesizer.

The compositions and methods of the invention can be used to, for example, rapidly identify and validate novel drug targets, to optimize the selection of the best drug candidates to move into clinical trials, to provide diagnostic information for drug development and patient management, and to detect and identify microorganisms.

Furthermore, one of the earliest applications of phage display technology was to search for new peptides that bind cell-surface receptors, primarily protein-binding cytokine receptors (Smith et al., Chem. Rev 97:391-410 (1997)). Targets used in such studies have expanded to include enzymes, intracellular signaling proteins, ion channels, cell surface architectures, and nonprotein targets. Since then, many types of ligands have been displayed on the surface of filamentous phage including peptides, antibody fragments, enzymes, protease inhibitors, transcription factors, cDNA libraries, cytokines, extracellular domain receptors, and protein scaffolds. Therefore, the compositions and methods of the invention can be especially useful for elucidating the complex network through which proteins interact with one another. Many signaling and structural proteins contain protein modules designed to mediate protein-protein interactions. Proteins that possess such interaction modules provide the molecular scaffold on which to organize macromolecular protein complexes. Because these domains bind to small linear peptide sequences, phage peptide display is a useful tool for identifying optimal binders and orphan ligands, for revealing the binding preferences of protein modules, and for providing direct proof of a biological model. Identification of the optimal binding substrate of orphan protein or protein interaction domains can also provide essential information for identification of their physiologically interacting substrates.

Data collected using the compositions and methods of this invention can be used to build a protein interaction map database. For example, regulated associations between select proteins are the basis of cellular signal transduction, and defining a signal transduction pathway can, in many cases, be reduced to tracing a chain of protein-protein interactions. Considering the large number of different protein interaction domains, a high throughput screen using the instant technology combined with bioinformatics tools provide an important data set of protein interactions. By indexing cell attachment an/or morphological changes, one can learn important information concerning cellular surface landscape and provide indexing profiles for cells in different physiological or pathological states.

### Phages and ALRs

An affinity ligand reagent (ALR) is a molecule that is recognized by a particular receptor. ALRs can be, for example, a polyclonal antibody, monoclonal antibody, single chain antibody (scFv), Fab fragment, F(ab′)₂ fragment, Fv fragment, a polypeptide, a small organic molecule, or combinations thereof An ALR of the invention can be expressed by a phage or phagemid. Methods of constructing phage or phagemid libraries comprising polyclonal antibodies, monoclonal antibodies, single chain antibodies (scFv), Fab fragments, F(ab′)₂ fragments, Fv fragments, small organic molecules, polypeptides, or combinations thereof are well known in the art.

Phage display is a technique where an ALR is genetically fused to a coat protein of a bacteriophage, for example M13, fd, f1, T7, T4 or lambda, resulting in display of the fused ALR on the exterior of the phage virion while the DNA encoding the fused ALR is contained within the virion. This physical linkage between the phage displayed ALR and the DNA encoding for it allows for screening of large numbers of ALRs.

For example, recombinant phage or phagemids can be used to produce libraries having about 10⁶-10⁸ ALRs (Smith, Science, 228:1315-7 (1985); Scott and Smith, Science, 249:386-390 (1990); Cwirla et al., Proc. Natl. Acad. Sci., 87:6378-6382 (1990); Devlin et al., Science, 249:404-406 (1990)). A phage vector comprises, for example, a bacteriophage replication origin, all functions required for viral propagation, and an antibiotic resistance gene. A phagemid vector comprises, for example, a plasmid replication origin, a bacteriophage replication origin, an antibiotic resistance gene, but no bacteriophage genes other than the gene fusion with the ALR to be displayed. Particles carrying phagemid DNA are produced by, for example, superinfecting a strain carrying the phagemid DNA in double-stranded plasmid form with an M13-like helper bacteriophage, which provides the bacteriophage functions necessary for the synthesis of circular single stranded DNA and bacteriophage coat. Phagemid vectors replicate as plasmids, but are packaged into bacteriophage particles when superinfected by helper bacteriophage. Phage and phagemid libraries can be constructed using methods well known in the art (Kay et al (1996) Phage Display of Peptides and Proteins: A Laboratory Manual, Academic Press, San Diego; Cortese et al., Curr. Opin. Biotechnol. 6:73-80 (1995); O'Neil et al., Methods Enzymol. 245:370-386 (1994); Hoogenboom et al., Immunotechnology, 4:1-20 (1998); Sarantopoulos et al., Comb. Chem. High Throughput Screen, 3:185-96 (2000) (see also, Ph.D.™ Display Peptide Technology, New England Biolabs, Beverly, Mass.; T7 Select™ Phage Display System (Novagen, Madison, Wis.)) and are also commercially available (see, e.g., Pre-Made T7 Select™ Libraries, Novagen; Ph.D.™ 7 Peptide 7-mer Library Kit and Ph.D.™ 12 Peptide 12-mer Library Kit, New England Biolabs).

The phage or phagemid vectors are prepared and ligated with target inserts and the resulting DNA is incubated such that the DNA is packaged into phage products. The target DNA inserts comprise amino acid coding regions. The sequence of the target DNA can comprise sequences found in nature, such as DNA sequences from a prokaryote or eukaryote, or can be sequences not found in nature (i.e., artificial sequences).

### Immobilizing Phages Expressing ALRs and ALRs to Solid Supports

Phages comprising ALRs and ALRs can be immobilized on a solid supports in predefined locations. A predefined location is a localized area on a surface of a solid support, which comprises one type of phage that expresses one type of ALR or one type of ALR. The predefined location can have any shape, e.g., circular, rectangular, elliptical, or wedge-shaped. A solid support can be any conceivable substrate. The solid support can be biological, nonbiological, organic, inorganic, or a combination of any of these, existing as particles, strands, precipitates, gels, beads, sheets, tubing, spheres, containers, capillaries, pads, films, plates, microarrays, biosensors, microtiter dishes, microfabricated sensors, or slides, for example. The solid support can be flat but can take on a variety of alternative surface configurations. For example, the substrate can contain raised or depressed regions. The solid support can be composed of any of a wide variety of materials, for example, polymers, plastics, resins, polysaccharides, silica or silica-based materials, fiberglass, latex, nitrocellulose, nylon, silicon wafers, magnetic beads, polystyrene, polyvinylchloride, carbon, metals, inorganic glasses, or porous or non-porous membranes.

Phages comprising ALRs and ALRs can be immobilized on a solid support by any means known in the art. For example, an ALR or phage can be immobilized on a solid support by noncovalent or covalent immobilization. Covalent immobilization can be accomplished by a direct linkage between a polypeptide and functional groups on the support or by a cross-linking agent. For example, covalent immobilization can be achieved by reacting the support with a bifunctional reagent, such as glutaraldehyde, that will react with both the support and a functional group, such as a hydroxyl or amino group on the phage or ALR. Further, a phage or ALR can be biotinylated and covalently immobilized on a solid support that has been pre-coated with, for example, streptavidin. A phage can be produced with a His tag (i.e., approximately 6 His residues) such that the phage binds to a solid support coated with nickel chealate. Additionally, phages or ALRs can absorb non-specifically to a support including, for example, plastic microtiter plates.

Further, ALRs can be chemically synthesized onto the solid support in predetermined positions as is known in the art. See, e.g., U.S. Pat. No. 5,143,854; U.S. Pat. No. 5,591,646; U.S. Pat. No. 5,744,305; U.S. Pat. No. 6,040,193; U.S. Pat. No. 6,040,423; U.S. Pat. No. 6,124,102.

At least about 2, 5, 10, 50, 100, 1,000, 10,000 or 100,000 different types of ALRs or phages specific for one biological construct or receptor can be present in a predefined location on a solid support. A solid support can also comprise ALRs or phages derived from at least about 2, 5, 10, 50, 100, 1,000, or 10,000 different biological constructs or receptors.

A predefined location in which a population of identical ALRs or phages is immobilized can be large enough in surface area to allow binding of about 1 or more, 10-100, or 100-10,000 biological constructs or receptors. However, the size of the location containing one type of ALR or phage is unlimited and solid supports comprising an area large enough for the binding of a large number of biological constructs or receptors is contemplated. An amount of a biological construct or receptor can bind to a location such that detection of the biological construct or receptor is possible and such that other adjacent locations are not obstructed by binding of the biological construct or receptor. The sizes of biological constructs and receptors are well known in the art or can be easily determined using methods well known in the art. The size of locations can be adjusted to accommodate smaller biological constructs or receptors (e.g. viruses) or larger biological constructs or receptors (e.g. parasites).

A type of phage is a phage that expresses one specific type of ALR. Each type of phage or ALR can be present on the solid support in a predefined location in multiple copies. In one embodiment of the invention, within a predefined location of the solid support, a phage population or ALR population can be substantially pure. That is, in some embodiments of the invention, predefined locations of the solid support contain phage populations that are at least about 50%, 75%, 90%, 95%, or 99% pure. The result is a non-uniform array of phages expressing ALRs or ALRs providing a variety of receptor binding sites of different specificity. Additionally, phages or ALRs can be immobilized on the solid support in adjacent, progressively differing concentrations to establish a gradient effect to, for example, titrate an antibody against an increasing amount of antigen.

Phages or ALRs can be applied to the solid support using any technique known in the art. For example, phages or ALRs can be applied to the solid support using an arrayer such as the Affymetrix® 417™arrayer.

### Methods of Detecting and Identifying Receptors

A phage array of the invention will have a variety of uses including, for example, screening large numbers of receptors for biological activity. To screen for biological activity, the solid support is exposed to one or more receptors, such as antibodies. The receptors can be labeled. The location of the label on the solid support is detected with, for example, photon detection or autoradiographic techniques. Through knowledge of the ALR at the predefined location where binding is detected, it is possible to quickly determine which ALR binds with the receptor and, therefore, the technique can be used to screen a large numbers of ALRs. Other applications of the invention include diagnostics in which various antibodies for particular receptors are used as ALRs and, for example, blood sera would be screened for immune deficiencies.

Any type of receptor can be detected and/or identified using the methods and compositions of the invention. A receptor is a molecule that has an affinity for a given ALR. Receptors can be naturally occurring or artificial molecules. Examples of receptors include, but are not limited to, a polyclonal antibody, a monoclonal antibody, a single chain antibody (scFv), a Fab fragment, a F(ab′)₂ fragment, a Fv fragment, antisera reactive with specific antigenic determinants (such as on viruses, cells or other materials), an agonist for a cell membrane receptor, an antagonist for a cell membrane receptor, a toxin, a hormone, a hormone receptor, an enzyme, an enzyme substrate, a cofactor, a drug, a lectin, a sugar, an oligonucleotide, a polypeptide, a small organic molecule, a prokaryotic cell, a eukaryotic cell, a cell membrane, a cell membrane receptor, an organelle, an oligosaccharide, or combinations thereof. A receptor can also be an outer coat polypeptide of a microorganism, such as bacteria, viruses, fungi, protozoa, and parasites.

The receptors can be purified or semi-purified or can be present in a sample such as a biological sample (including, for example, saliva, sputum, blood, urine, feces, cerebrospinal fluid, amniotic fluid, wound exudate, or tissue), a water sample, an environmental sample, a pharmaceutical sample, or a food sample.

The receptors to be identified or detected can be labeled before or after they are applied to the solid support. Receptors can be labeled by any method known in the art including, for example, the use of a colormetric label, a fluorescent label, a biolumnescent label, a chemiluminescent label, a magnetic particle label, a radioactive label, a labeled antibody specific for a receptor, quantum dot, and a fluorescent particle label. Alternatively, the receptors are not labeled and are detected by, for example, examining modification of mass, refractive index, or surface roughness of the solid support once the receptors are bound to the solid support.

Typically, a purified sample or a sample suspected of comprising one or more receptors is applied to the solid support comprising at least about two types of phage under conditions that allow binding of the receptor to the phage-expressed ALRs present on the solid support. A wash step can be employed to remove any unbound receptors or sample debris. The immobilized receptors are detected and optionally identified by comparison to known binding patterns of known receptors. The solid support can be scanned or read by any method known in the art. For example, by using the Affymetrix® 418™ or 428™ array scanners.

The solid support with bound, labeled receptors is placed in, for example, a microscope detection device for identification of the predefined location or locations where binding takes place. The microscope detection device includes, for example, a monochromatic or polychromatic light source for directing light at the solid support, means for detecting fluoresced light from the substrate, and means for determining a location of the fluoresced light. The means for detecting light fluoresced on the solid support can be, for example, a photon counter. The means for determining a location of the fluoresced light can include an x/y translation table for the solid support. Translation of the slide and data collection are recorded and managed by an appropriately programmed digital computer. Therefore, for a solid support that has a matrix of phages expressing ALRs on its surface, it is possible to determine which of the ALRs specifically binds to a fluorescently marked receptor.

Therefore, the compositions and methods of the invention can be used to identify the presence or absence of a receptor for a specific ALR. Furthermore, the methods and compositions of the invention can be used to detect the relative binding affinity of ligands to a variety of ALRs. It is likely that a receptor will bind to several ALRs on an array, but will bind much more strongly to some ALRs than others. Strong binding affinity is demonstrated by a strong signal, such as a fluorescent signal, because many ligand molecules will bind in a region of a solid support comprising a strongly bound ALR. Alternatively, a weak binding affinity is demonstrated by a weak signal due to the relatively small number of receptor molecules that bind in a particular region of a solid support having an ALR with a weak binding affinity for the receptor. Therefore, it is possible to determine relative binding affinity or avidity of an ALR by examining the intensity of a signal in a region containing that ALR. Quantitative data on affinities can be obtained by varying washing conditions and concentrations of the receptor. A comparison can then be made to known ALR-receptor pairs.

One embodiment of the invention provides a receptor screening system comprising a reader instrument for detecting signals, such as fluorescent signals, from reporter molecules immobilized on the solid support, a digital detector for receiving data from the reader instrument, and a computer device for receiving and processing digital data from the detector.

Another embodiment of the invention provides a method for immobilizing many different “bait” proteins onto different locations of a microarray surface. Methods of immobilizing proteins or polypeptides onto a microarray surface are well known in the art. The microarray surface is exposed to a sample solution containing a phage display library. Phages within the sample solution will selectively bind to microarray locations that contain high affinity bait proteins, thus creating a phage microarray where individual phage clones have bound to selected binding partners on the solid surface. The microarray is rinsed to remove all nonbonding phages. The identities of the bound phages is determined by separating them from the microarray surface, and sequencing the portion of their DNA that encodes the cognate outer surface protein. Ideally, the bound phages can be separated from individual microarray locations so a one-to-one correspondence between the bait protein and its phage display library binding partner can be determined.

Because phage concentration can be readily amplified through infection of a host strain bacteria, only a single phage need be extracted from the microarray surface and grown in culture media. Methods for selecting and extracting bound phage from the microarray can include, for example: 1. Coverage of the microarray surface with adhesive media (such as culture media) and extraction of material from the microarray using a conventional bacterial plaque picking robot. 2. Immersion of the microarray surface in a liquid medium and application of an electric field to selected microarray locations so that bound phage from only one array location are repelled from the surface and into the liquid medium, which is collected for phage concentration amplification.

### Biological Construct Identification by Outer Coat Protein Fingerprinting

The present invention also provides a rapid, non-culture based detection, identification, and quantification system for samples including, for example, biological, environmental, pharmaceutical process stream, food, and water samples. The method utilizes unique molecular biology techniques coupled to an intelligent information system that “fingerprints” biological constructs such as prokaryotic cells, eukaryotic cells, bacteria, virus, fungus, protozoa, and parasites throughout the repertoire of molecules including proteins, sugars, lipids, and other distinct molecular species that are displayed on their outer surface. The fingerprint is measured throughout the biological construct's affinity with a collection of ALRs that are “panned” from a phage display library, and placed onto a solid support for measuring the interaction between unknown samples and the ALRs, such as a microarray biosensor chip. ALRs or phages expressing ALRs are immobilized on a solid support in a predefined location. Pattern recognition algorithms are trained to recognize the response pattern of biological constructs or receptors to the ALRs, and to provide identification based on many parallel positive and negative tests.

Because most biological construct surface components have not been defined, and those that have been defined are not specific enough to a particular biological construct to distinguish between, for example, strains or similar species, there is a major advantage to defining a biological construct's fingerprinting based on the binding characteristics of multiple ligands. For example, a microarray format makes possible the resolution of complex binding patterns of ligands with varying affinities for the biological construct's surface components.

### Biopanning

Once a library of ALRs is obtained, the library can be biopanned against a biological construct of interest. The result of biopanning is the identification of a set ALRs that specifically bind to a biological construct of interest. For example, each type of biological construct, including different strains of the same biological construct, will have a different set of specifically binding ALRs.

Biological constructs that can be biopanned in order to identify sets of specifically binding ALRs include, for example, prokaryotic cells, eukaryotic cells, bacteria, viruses, fungi, protozoa, parasites, and prions. Specific examples include, but are not limited to Candida, Aspergillus, Sporothrix, Blastomyces, Histoplasma, Cryptococcus, Pneumocystis, Coccidioides, Tinea, Toxoplasma, Plasmodium, Pseudomonas, Actinobacillus, Staphylococcus, Bacillus, Clostridium, Listeria, Corynebacterium, Actinomyces, Mycoplasma, Nocardia, Bordetella, Brucella, Francisella, Legionella, Enterobacter, Escherichia, Klebsiella, Proteus, Salmonella, Shigella, Streptococcus, Yersinia, Vibrio, Campylobacter, Helicobacter, Bacteroides, Chlamydia, Borrelia, Treponema, Leptospira, Aeromonas, Rickettsia, Ascaris, Cryptosporidium, Cyclospora, Entamoeba, Giardia, Shistosoma, Trypanosoma, herpes virus, cytomegalovirus, Epstein-Barr virus, hepatitis virus, adenovirus, papillomavirus, polyomavirus, enterovirus, rotavirus, influenza virus, paramyxovirus, rubeola virus, rhabdovirus, human immunodeficiency virus, arenavirus, rhinovirus, and reovirus.

In biopanning, an entire combinatorial library of phage expressing ALRs such as polyclonal antibodies, monoclonal antibodies, single chain antibodies (scFv), Fab fragments, F(ab′)₂ fragments, Fv fragments, small organic molecules, polypeptides, or combinations thereof, is incubated with a biological construct of interest. Phages that bind to the biological construct with high affinity are separated from the mixture and amplified by, for example, propagation in a host cell such as E. coli. Phages that bind to the biological construct can be separated by methods well know in the art including, for example, centrifugation or elution. Bacteria with bound phage attached can generally be separated from unbound phage by spinning in a centrifuge at 6000 rpm for 10 minutes. The heavier bacteria will form a pellet in the bottom of the centrifuge tube, and the unbound phage in the supernatant are separated by pouring them away. The pellet can be resuspended and centrifuged several times to improve the quality of the separation process. This procedure of binding and separation can be repeated, for example, for three to five cycles, until a manageable number, for example, less than 1,000 or 100, of the highest affinity phage can be isolated from the library. The individual phage that have been selected by virtue of their binding characteristics to the biological construct are amplified. The result is a large number of separate phages bearing ALRs that are specific for a first biological construct of interest.

If desired, the ALR portion of the phage can be cloned, sequenced, expressed in a host organism, and purified. The ALR can then be used independently of the phage.

The library can be biopanned against additional individual types of biological constructs and additional individual strains or variants of biological construct. Furthermore, additional libraries can be biopanned against the different types of biological constructs. The biopanning can be done, for example, for all the different biological construct species to be tested in a given environmental or biological sample thus creating an array for a particular use (e.g., blood testing or urinary tract infection testing). Once several biological constructs have been biopanned against one or more libraries, a number of ALRs having different binding specificities for different biological constructs can be selected so that biological constructs can be identified and differentiated using the methods of the invention. An ALR can be screened for binding characteristics to at least about 2, 10, 50, 100, or 1,000 different biological constructs.

### Methods of Detecting and Identifying Biological Constructs

Any type of biological construct can be identified using the methods and compositions of the invention. For example, prokaryotic cells, eukaryotic cells, bacteria, viruses, fungi, protozoa, and parasites can be detected and identified. Further, different stains or variants of the same biological construct can be differentiated and identified.

The biological constructs can be in a pure culture or can be present in a sample such as a biological sample (including, for example, saliva, sputum, blood, urine, feces, cerebrospinal fluid, amniotic fluid, wound exudate, or tissue, from an infected individual), a water sample, an environmental sample, pharmaceutical, or a food sample.

The biological constructs to be identified or detected can be labeled before or after they are applied to the solid support. Biological constructs can be labeled by any method know in the art including, for example, the use of colormetric dyes, fluorescent dyes, bioluminescent dyes, chemiluminescent dyes, magnetic particle dyes, labeled antibodies, radioactive labels, quantum dot, and fluorescent particle labels. Alternatively, the biological constructs are not labeled and are detected by, for example, gravimetric sensing (Prusak-Sochaczewski et al., Enzyme Microb. Technol., 1990, vol. 12, March), change in refractive index, change in surface roughness of support, or phase contrast microscopy. Methods that detect a modification in mass, refractive index or surface roughness of the solid support can also be used to detect the bound biological constructs.

A sample suspected of comprising biological constructs can be applied to a solid support under conditions that allow binding of a biological construct to one or more ALRs present on the solid support. A wash step can be employed to remove any unbound biological constructs or sample debris. The immobilized biological constructs are detected and identified by comparison to know binding patterns of biological constructs. Bound biological constructs can be identified as described above for receptors.

### Methods of Quantifying Biological Constructs

The quantity of biological constructs present in a sample can be determined by measuring the density of surface-adsorbed biological constructs for each ALR. The density can be measured by actually counting the adsorbed biological constructs for each ALR using, for example, a phase contrast microscope. However, one embodiment of the invention provides for the measurement of a signal from each predetermined location of a solid support. The signal can be a fluorescence emission, optical density, or radioactivity, for example. The signal measured from each predetermined location can integrate the signal from all the material in the location to give an overall population average of all of the molecules at a location. Alternatively, the signal data can be taken over regions smaller than the area dedicated to one type of ALR. Using this technique, a number of data points can be collected for each predefined location and an average of the data points can be determined.

The amount of signal detected for each ALR predetermined location can be compared to amount of signal generated by know amounts of a particular biological construct when applied to each ALR predetermined location.

### Computer Systems

### Exemplary Experimental Data Processing System

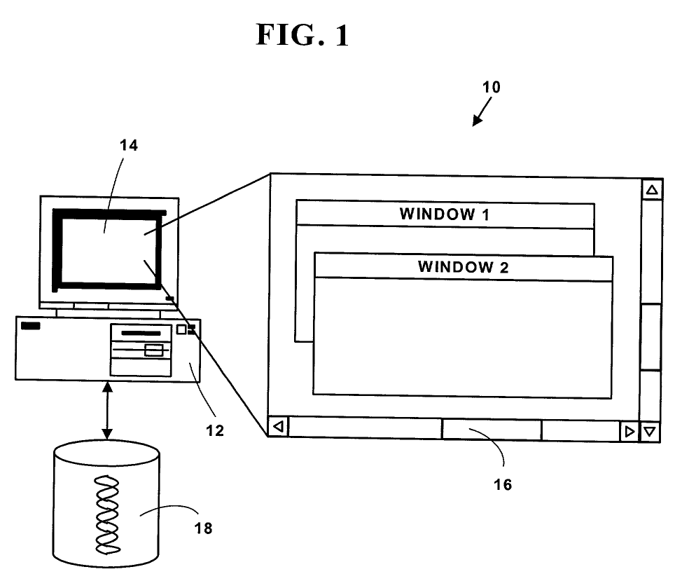

FIG. 1 is a block diagram illustrating an exemplary experimental data processing system for one exemplary embodiment of the present invention. The experimental data processing system (10) includes a computer (12) with a computer display (14). The computer display presents a windowed graphical user interface (“GUI”) (16) to a user. A database (18) includes experimental information. The database can be integral to a memory system on the computer or in secondary storage such as a hard disk, floppy disk, optical disk, or other non-volatile mass storage devices.

An operating environment for the data processing system for one embodiment of the present invention include a processing system with one or more speed processors and a memory. The processor can be electrical or biological. In accordance with the practices of persons skilled in the art of computer programming, the present invention is described below with reference to acts and symbolic representations of operations or instructions that are performed by the processing system, unless indicated otherwise. Such acts and operations or instructions are referred to as being “computer-executed” or “processor executed.”

It will be appreciated that acts and symbolically represented operations or instructions include the manipulation of electrical signals or biological signals by the processor. An electrical system or biological system represents data bits which cause a resulting transformation or reduction of the electrical signals or biological signals, and the maintenance of data bits at memory locations in a memory system to thereby reconfigure or otherwise alter the processor's operation, as well as other processing of signals. The memory locations where data bits are maintained are physical locations that have particular electrical, magnetic, optical, or organic properties corresponding to the data bits.

The data bits can also be maintained on a computer readable medium including magnetic disks, optical disks, organic memory, and any other volatile (e.g., Random Access Memory (“RAM”)) or non-volatile (e.g., Read-Only Memory (“ROM”)) mass storage system readable by the processor. The computer readable medium includes cooperating or interconnected computer readable medium, which exist exclusively on the processing system or be distributed among multiple interconnected processing systems that can be local or remote to the processing system.

### Analyzing Binding Pattern and Binding Intensity Data

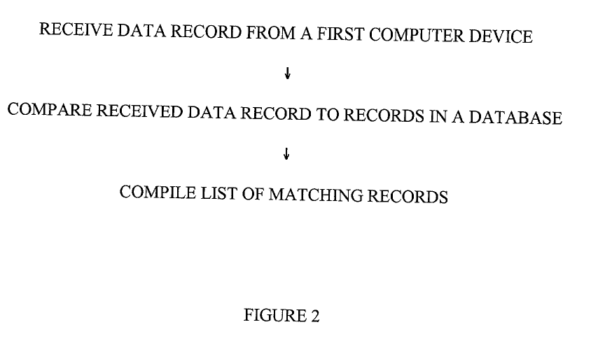

In one exemplary embodiment of the present invention, a data record such as an ALR binding pattern record, an ALR binding intensity record, or both can be received from a first computer device (FIG. 2). ALR binding patterns are the ALRs of a particular array of ALRs that are bound by a specific biological construct, mixture of biological constructs, receptor, or mixture of receptors. An ALR can be immobilized on a support or can be expressed by a phage immobilized on a support. ALR binding intensities are the concentrations or amounts of binding of a specific type of biological construct to a particular type of one or more ALRs. The received information is compared to records in a database and a list of matching information is compiled.

Databases of the invention comprise records of ALR binding patterns of particular biological constructs or receptors to a particular ALR or an ALR group or array. For example, a database record can comprise information regarding which ALRs of an array of ALRs that a specific type of biological constructs or receptors binds. Databases of the invention can further comprise ALR binding intensities of biological constructs or receptors to a particular array when a specific concentration or amount of biological constructs or receptors is applied to the array. Database records can also comprise information on which ALRs are bound by a mixture of two or more types of biological constructs or receptors .

In order to generate a database record, a group or one type of biological construct or receptor, such as a particular strain of Borrelia burgdorferi, is tested against a specific ALR or a specific group or array of ALRs and the results (i.e., binding or no binding) are recorded as a database record. Further, different amounts or concentrations of the biological construct or receptor can be applied to a specific ALR or a specific group or array of ALRs and the intensity of binding of the different amounts or concentrations is recorded as a database record. For example, a label such as a fluorescent label can be incorporated into target biological constructs, receptors, or unbound ALRs for detection by, for example, laser-induced fluorescence, which is used to obtain data. However, other labels and other detection methods can be used to generate the data records of the invention.

An information signal based on indicated fluorescence intensities of the label is included in a resulting experimental data file as digital data. The information signal includes raw label fluorescence intensities. Label responses are relatively broadband spectrally and typically include spectral overlap. In one embodiment of the present invention, spectral overlap is removed and a normalized baseline can be created with a combination of filtering techniques. The result is a data record and one or more data records of the invention are compiled into a database.

The invention therefore provides a method of providing information concerning biological construct or receptor ALR binding patterns to a first computer device through a second computer device. A computer device can be for example, a web server, a stand alone computer, or a personal digital assistant. Optionally, the first and second computer devices are connected by a network. At least one ALR binding pattern is received from a first computer device. The ALR binding pattern can also comprise binding intensity data for one or more ALRs. The at least one ALR binding pattern received from a first computer device is compared to records in an ALR binding pattern information database, and a list of matching biological construct or receptor information from the records of an ALR binding pattern information database matching the at least one ALR binding pattern received from a first computer device is compiled. The list can optionally also provide matching records for ALR binding intensities if this information was provided by the first computer device. The list can be visually displayed on a display device.

The step of comparing can comprise obtaining a binding pattern from the first computer device, obtaining a binding pattern from the database, and comparing these binding patterns. These steps are repeated until a match is made. Alternatively, these steps can be repeated until all binding patterns in the database are compared to the binding pattern from the user.

The step of comparing can comprise obtaining a binding pattern from the first computer device, obtaining a binding pattern from the database, and comparing these binding patterns. These steps are repeated until a match is made. Alternatively, these steps can be repeated until all binding patterns in the database are compared to the binding pattern from the user.

Numerous algorithms can be developed to perform such comparisons. Let X=(x1, x2, . . . , xn) denote the response of the n ALRs to a homogeneous sample comprised, for simplicity, of a single biological construct. To determine the identity of this biological construct, one embodiment is to compare X against a database of responses for these same ALRs when exposed to a variety of different biological constructs. Let k denote the number of distinct biological constructs tabulated in the database for this same set of ALRs. The database can be represented by the set of class means (centroids) for each such biological construct, μ1, μ2, . . . , μk. The identity of X is then determined by first computing the error term:

Ei=|μi−X|,i=1, . . . , k

and subsequently finding the biological construct that minimizes this error. An alternative formulation is to compute the correlation or dot product between μi and X, and selecting the biological construct that yields the highest correlation or dot product.

Yet another embodiment, when the number of ALRs is high, is to use either regression analysis or principal components analysis to reduce the dimensionality of the comparison to only these ALRs that are most relevant (see e.g., Dillon and Goldstein, Multivariate Analysis, Wiley, 1984, NY).

When the sample contains a mixture of biological constructs, repeated iterations of the above procedure can be utilized. Alternatively, multi-discriminate analysis, factor analysis, clustering, or neural network (including self organizing network) algorithms can also be used.

Also contemplated by the invention is a system allowing users to obtain information on the identity or quantity of biological constructs or receptors in a sample from a directory available via a computer. The computer comprises, in combination, a second computer device in communication with a first computer device to allow users to enter selection criteria for retrieving records of biological construct or receptor ALR binding patterns and binding intensities and a database. The database comprises records of biological construct or receptor ALR binding patterns and binding intensities. The second computer device produces a list of matching ALR binding pattern and binding intensity records that match the selection criteria and displays the matching patterns and binding intensities on the list in an order determined by each matching biological construct's or receptor's similarity to the selection criteria. Optionally, the first and second devices can be connected by a network.

Selection criteria can comprise an ALR binding pattern, an ALR binding intensity, or an ALR binding pattern and an ALR binding intensity. The ALR binding pattern and intensity provided by the user is determined by applying a sample suspected of containing biological constructs or receptors to a solid support comprising ALRs specific for at least one type of biological construct or receptor. The solid support comprises at least X number of different ALRs capable of specifically binding to each type of biological construct or receptor bound to the solid support in predefined locations. The number X includes, for example, 5, 10, 50, 100, or 1,000. The list can be visually displayed on a display device.

The following are provided for exemplification purposes only and are not intended to limit the scope of the invention described in broad terms above. All references cited in this disclosure are incorporated herein by reference.

### EXAMPLES

### Example 1

### Phage Microarray

In order to demonstrate that phage particles expressing ALRs can be used effectively on a microarray, a phage microarray was implemented for identification of a microorganism. This example demonstrates that a phage microarray is useful for microorganism detection by showing that phage with a high affinity constant against the outer surface of intact microorganisms can be panned from a large combinatorial phage display library, that the panned phage can be adhered to the surface of a microarray, and that when exposed to a solution containing microorganisms, the microarray locations containing phage have the ability to selectively gather target microorganisms while excluding non-target microorganisms.

For this example, Lyme disease bacteria Borrelia burgdorferi (strain NECK) was selected as the target microorganism. A commercially available (New England BioLabs PhD-7) 7-mer linear peptide phage display library containing over 5×10⁹ individual phage clones was used. The PhD-7 library contains filamentous M13 bacteriophage in which the differential peptide is expressed by the P3 gene, resulting in expression of 5 peptide copies on the phage outer coat. The host strain for M13 phage is E. coli (strain ER2738, supplied with New England BioLabs Phage Display Library kit).

In order to obtain high affinity binding phage clones from the library, the library was panned using the following procedure: First, the target microorganism (NECK) and the full library were mixed together and allowed to incubate (room temperature, 1 hour, with agitation). The co-incubation enables the phage clones to compete for binding sites on the outer surface of the NECK, so that only the highest affinity phage clones will be adhered to bacteria. After incubation, the bacteria with attached phages are separated from the remainder of the unbound phage library by microcentrifugation (11,000×g, 4° C., 10 min). The unbound supernatant phages are poured off, and the bacteria with bound phage are resuspended and washed three times in phosphate buffer solution. The phages are separated from the bacteria by reducing the pH of the solution (pH 2.2 obtained with a 0.2M glycine-HCl/1.0 mg/ml BSA solution). The eluate was immediately neutralized after separation (1M tris-HCl pH 9.1). Following the phage library manufacturer's instructions, the phage clones are amplified using the host strain E. coli to increase their titer to approximately 10¹¹ phage/ml. Following this first round of panning, the number of clones has been down-selected by several orders of magnitude, but the resulting sublibrary, denoted as PhD-7.1, contains many clones of very low affinity constant. In order to isolate the highest affinity clones, the library panning procedure (bacteria incubation, centrifugation, phage separation, and amplification) is repeated several times. In this experiment, the panning procedure was performed three times to produce sub-libraries PhD-7.2 and finally PhD-7.3.

Individual phage clones from sub-library PhD-7.3 were isolated following the phage library manufacturer's instructions by reducing the titer, and growing easily separated phage colonies in a culture plate containing host strain E. coli and agar nutrient. The individual colonies were picked from the culture plate, and individually suspended in phosphate buffer solution. Sixty-five randomly selected colonies were picked, and the phage clones were amplified to an approximate titer of 1×10¹² phage/ml. The individual clones were designated as PhD-7.3.1 through PhD-7.3.65; the nonbonding supernatant phage was also saved, and designated PhD-7.3.SN.

As a positive control to test the ability of panned phage to bind NECK, the blood serum of an individual with an active Lyme disease infection was obtained. The serum contains a high concentration of high affinity polyclonal antibodies against NECK, as verified by ELISA assay. The serum most likely represents the highest affinity binding reagent for NECK that is available. As a negative control, the blood serum of a healthy individual was obtained. The healthy individual had no antibodies against NECK as verified by ELISA assay. The phage supernatant does not have high affinity for NECK, and also serves as a negative control.

Phage clones PhD-7.3.1 through PhD-7.3.10 and the supernatant PhD-7.3.SN were placed into the wells of a 96-well microwell plate at their highest titer (˜1×10¹² clones/ml). The undiluted positive and negative control sera were also placed into the microwell plate.

A microarray was prepared by dispensing small volumes of each test solution onto individual locations on a glass slide. A commercially available microarray spotter (Affymetrix) was used to draw solutions from the 96-well plate, and apply droplets of ˜40 picoliters onto a glass slide in linear array configuration. For each solution, 10 replicate spots were deposited. Spot diameter is approximately 150 micrometers. The glass slide (Cel Associates) is coated with glutaraldehyde to facilitate the nonreversible immobilization of phage or proteins. Three copies of the microarray were produced. In order to block nonspecific binding of material to locations on the slide not containing a reagent, the slides were soaked in a phosphate buffer solution containing 0.5% bovine serum albumin for 15 minutes.

The microarrays were exposed to analyte solutions containing fluorescently stained microorganisms. For this experiment, three types of stained microorganisms were prepared: 1). Lyme disease bacteria (strain NECK) 2). E. coli with the ability to be infected by M13 phage (Fl+), and 3). E. coli without the ability to be infected by M13 phage (Fl−). The Fl+ is intended as a positive control for exposure of the microarray to a microorganism, while the Fl− (strain W4680, reference J. Lederberg and Cook, Genetics 47, p. 1335-1353, 1962) is a negative control that should bind minimally to any of the phage. The bacteria were stained by exposing them to a solution containing Nile Red dye in phosphate buffer solution (1:1000 dilution) for 15 minutes. To assure that the analyte solution contains only stain that is trapped within the bacteria, the stained organisms were “washed” by separating the bacteria from the stain solution with a centrifuge, pouring off the solution, and resuspending the bacteria in buffer solution. The washing procedure was repeated three times. The final analyte solutions of NECK, Fl+, and Fl− contained 2×10⁹ cfu/ml.

Each of the three analyte solutions were incubated with a separate microarray slide by exposing the microarray to 2 ml of solution for 1 hour. After the exposure, the slides were rinsed in buffer solution, rinsed in distilled water, and dried.

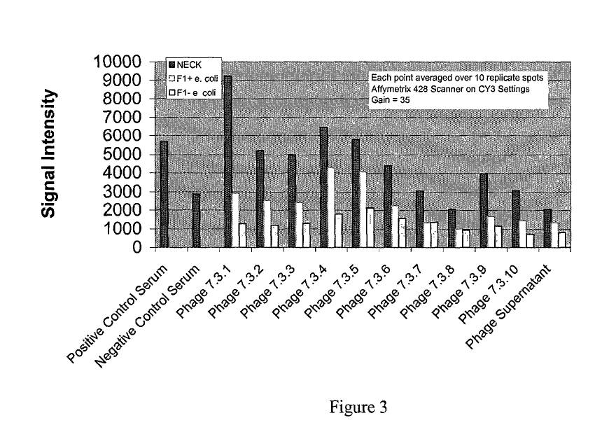

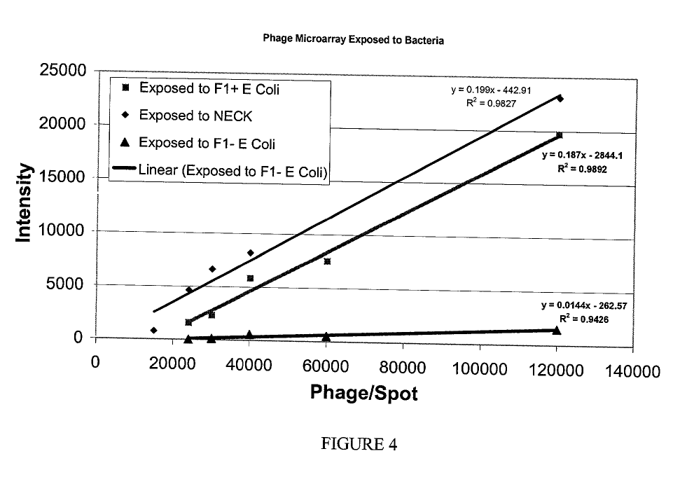

After drying, fluorescence images of the slides were obtained using a commercially available scanning confocal microscope (Affymetrix 428™ scanner) with a laser excitation wavelength of 532 nm and an emission filter with a bandpass centered at 570 nm. The integrated intensity of the fluorescence signal from each microarray location was recorded, and averaged over 10 replicate spots. The resulting signal intensity for each combination of microarray reagent and microorganism is shown in FIG. 3. A dose-response curve for one of the ALRs in this experiment is shown in FIG. 4, in which the density of phage within a microarray spot is varied. FIG. 4 shows that greater concentration of phage within the microarray spot is advantageous for discriminating between different microorganisms.

The data show that several of the panned phage have affinity for NECK that is, nearly as high as the positive control serum, and that the supernatant phage have low affinity for the NECK. This shows that the library panning procedure for selecting high affinity binding phage clones was successful. These data also indicate that phage clones expressing a binding protein for a microorganism can be effectively utilized as a reagent in a microarray. Confidence in the binding properties of the NECK to the panned phage is increased by the negative controls (phage supernatant and negative control serum), which do not display a high affinity binding signal. Furthermore, the ability of the positive control analyte, Fl+E. coli to bind the phage microarray with higher affinity than the negative control, Fl−E. coli, demonstrates the specificity of the assay This simple phage microarray is able to distinguish between NECK and Fl−E. coli based on the interactions of the phage with the outer surface protein components of the microorganisms. Thus, a phage microarray can discriminate between two different microganisms because microarray locations containing a high affinity phage clone to one organism have the ability to selectively exclude the binding of an organism that does not have high affinity.

### Example 2

### Differentiation of Borrelia Strains

In vitro cultivated strains of Borrelia N-40, which expresses abundant amounts of outer surface protein A (OspA), and Borrelia 7×297, which does not express OspA, were used as a model system. An affinity ligand reagent monolayer was immobilized on a solid support through sequential exposure to solution containing avidin, biotinylated human IgG, and human anti-OspA. When the sensor was challenged by the presence of 10⁷ Borrelia 7×297, no significant microbial adhesion was observed since the intended recognition protein, OspA, was absent from the bacteria. Meanwhile, when the surface was exposed to the same amount of Borrelia N-40, a significant number adherent bacteria were observed by fluorescence microscopy, which illustrates that the surface specifically detected the presence of N40.

This example demonstrates that is possible to differentiate microorganisms based upon differences in distinct molecular species on their outer surface. While this example utilizes a visual observation to measure the deletion of a single outer surface component, a microarray biosensor with a library of ALRs for outer surface components will enable detailed fingerprinting of individual microbial species.

We claim:

1. A solid support comprising at least about ten types of phages, wherein each phage displays a different affinity ligand reagent (ALR), and wherein each type of phage is immobilized to the solid support in a predefined location.

2. The solid support of claim 1, wherein there are at least about 100 types of phages.

3. The solid support of claim 1, wherein there are at least about 1,000 types of phages.

4. The solid support of claim 1, wherein the ALR is selected from the group consisting of a polyclonal antibody, a monoclonal antibody, a single chain antibody (scFv), a Fab fragment, a F(ab′)₂ fragment, a Fv fragment, a polypeptide, and a small organic molecule.

5. A method of detecting at least one receptor in a sample comprising:
applying the sample to a solid support comprising at least about two types of phages wherein each phage displays a different affinity ligand reagent (ALR), wherein each type of phage is immobilized to the solid support in a predefined location; and detecting the at least one receptor bound to the solid support.

6. The method of claim 5, further comprising identifying which phages on the solid support bind the at least one receptor.

7. The method of claim 5, wherein the solid support is washed before detection of the at least one receptor.

8. The method of claim 5, wherein the receptor is selected from the group consisting of a polyclonal antibody, a monoclonal antibody, a single chain antibody (scFv), a Fab fragment, a F(ab′)₂ fragment, a Fv fragment, antisera, an agonist for a cell membrane receptor, an antagonist for a cell membrane receptor, a toxin, a hormone, a hormone receptor, an enzyme, an enzyme substrate, a cofactor, a drug, a lectin, a sugar, an oligonucleotide, a polypeptide, a small organic molecule, a prokaryotic cell, a eukaryotic cell, a cell membrane, a cell membrane receptor, an organelle, and an oligosaccharide.

9. The method of claim 5, wherein the receptor is an outer coat polypeptide of a microorganism selected from the group consisting of bacteria, viruses, fungi, protozoa, and parasites.

10. The method of claim 5, wherein the ALR is selected from the group consisting of a polyclonal antibody, a monoclonal antibody, a single chain antibody (scFv), a Fab fragment, a F(ab′)₂ fragment, a Fv fragment, a small organic molecule, and a polypeptide.

11. The method of claim 5, wherein the solid support comprises at least about 100 types of phages each displaying a different ALR.

12. The method of claim 5, wherein the at least one receptor is labeled before application to the solid support.

13. The method of claim 12, wherein the label is selected from the group consisting of a colormetric label, a fluorescent label, a biolumnescent label, a chemiluminescent label, a magnetic particle label, a radioactive label, a labeled antibody specific for a receptor, quantum dot, and a fluorescent particle label.

14. The method of claim 5, wherein the at least one receptor is labeled after application to the solid support.

15. The method of claim 14, wherein the label is selected from the group of labels comprising a colormetric label, a fluorescent label, a biolumnescent label, a chemiluminescent label, a magnetic particle label, a radioactive label, quantum dot, a labeled antibody specific for a receptor, and a fluorescent particle label.

16. The method of claim 5, wherein detection of the receptor is accomplished by a method selected from the group consisting of detecting modification of mass, detecting refractive index, and detecting surface roughness of the solid support.

17. A method of preparing an array of affinity ligand reagents (ALRs) comprising the steps of:
(a) incubating a combinatorial library comprising ALRs with a first biological construct, wherein the biological construct is selected from the group consisting of a cell, bacteria, virus, fungus, protozoa, and parasite;
(b) separating ALRs that bind to the biological construct;
(c) immobilizing multiple copies of each ALR of step (b) to a solid support in a predefined location;
(d) repeating steps (a)-(b) at least once with at least one additional biological construct and immobilizing each specific ALR to a predefined location on the solid support of step (c);
wherein an array of ALRs is prepared.

18. The method of claim 17, further comprising identifying the ALR of step (b).

19. The method of claim 17, wherein the ARLs are synthesized on the solid support.

20. The method of claim 17, wherein the combinatorial library is selected from the group of libraries consisting of polyclonal antibodies, monoclonal antibodies, single chain antibodies (scFv), Fab fragments, F(ab′)₂ fragments, Fv fragments, small organic molecules, and polypeptides.

21. The method of claim 17, wherein at least about 10 different ALRs specific for each biological construct are immobilized on the solid support.

22. The method of claim 17, wherein the solid support comprises ALRs derived from at least about 10 different biological constructs.

23. The method of claim 17, wherein the solid support is selected from the group consisting of glass, nitrocellulose, nylon, silicon wafers, microfabricated sensors, polystyrene, and polyvinyl chloride.

24. The method of claim 17, comprising the additional step of determining binding characteristics of each ALR of step (b) to at least about 10 different biological constructs.

25. A method of preparing an array of phages comprising affinity ligand reagents (ALRs) comprising the steps of:
(a) incubating a phage combinatorial library comprising ALRs with a biological construct, wherein the biological construct is selected from the group consisting of a cell, bacteria, virus, fungus, protozoa, and parasite;
(b) separating phages that bind to the biological construct;
(c) amplifying one or more phages of step (b);
(d) immobilizing each specific phage of step (c) to a solid support in a predefined location;
(e) repeating steps (a)-(c) at least once with at least one additional biological construct and immobilizing each phage to a predefined location on the solid support of step (d); wherein an array of phages comprising ALRs is prepared.

26. The method of claim 25, wherein the ALRs are selected from the group consisting of polyclonal antibodies, monoclonal antibodies, single chain antibodies (scFv), Fab fragments, F(ab′)₂ fragments, Fv fragments, small organic molecules, and polypeptides.

27. The method of claim 25, wherein at least about 10 different phages specific for each biological construct are attached to the solid support.

28. The method of claim 25, wherein the solid support comprises specific phages derived from at least about 10 different biological constructs.

29. The method of claim 25, wherein the solid support is selected from the group consisting of glass, nitrocellulose, nylon, silicon wafers, microfabricated sensors, polystyrene, and polyvinyl chloride.

30. The method of claim 25, wherein steps (a) and (b) are repeated at least one time for each biological construct.

31. The method of claim 25, comprising the additional step of determining binding characteristics of each phage of step (c) to at least 10 different biological constructs.

32. A method of preparing an array of affinity ligand reagents (ALRs) comprising the steps of:
(a) incubating a phage combinatorial library comprising ALRs with a biological construct, wherein the biological construct is selected from the group consisting of a cell, bacteria, virus, fungus, protozoa, and parasite;
(b) separating phages that bind to the biological construct;
(c) cloning one or more phages of step (b);
(d) isolating the ALR portion of each phage of step (c);
(e) immobilizing the ALRs of step (c) to a solid support in a predefined location;
(f) repeating steps (a)-(d) at least once with at least one additional biological construct and immobilizing each ALR to a predefined location on the solid support of step (e);
wherein an array of ALRs is prepared.

33. The method of claim 32, wherein the ALRs are chemically synthesized on the solid support.

34. The method of claim 32, wherein the ALRs are selected from the group consisting of polyclonal antibodies, monoclonal antibodies, single chain antibodies (scFv), Fab fragments, F(ab′)₂ fragments, Fv fragments, small organic molecules, and polypeptides.

35. The method of claim 32, wherein at least about 10 different ALRs specific for each biological construct are immobilized on the solid support.

36. The method of claim 32, wherein the solid support comprises ALRs derived from at least about 10 different biological constructs.

37. The method of claim 32, wherein the solid support is selected from the group consisting of glass, nitrocellulose, nylon, silicon wafers, microfabricated sensors, polystyrene, and polyvinyl chloride.

38. The method of claim 32, wherein steps (a) and (b) are repeated at least one time for each biological construct.

39. The method of claim 32, comprising the additional step of determining binding characteristics of each ALR of step (d) to at least about 10 different biological constructs.

40. A method of identifying one or more biological constructs in a sample suspected of containing biological constructs comprising the steps of:
(a) applying the sample to a solid support comprising phages comprising ALRs or ALRs, wherein the ALRs are specific for at least two different biological constructs, wherein at least about 10 different ALRs capable of specifically binding to each of the at least two different biological constructs or at least about 10 different phages comprising ALRs capable of specifically binding to each of the at least two different biological constructs are bound to the solid support in predefined locations;
(b) detecting biological constructs bound to the ALRs; whereby a biological construct is identified in the sample.

41. The method of claim 40 further comprising the step of detecting the amount of one or more biological constructs bound to the ALRs, whereby a biological construct is quantified in the sample.

42. The method of claim 40, wherein the solid support comprises ALRs specific for at least about 10 different biological constructs.

43. The method of claim 40, further comprising the step of washing the solid support before detecting the biological constructs.

44. The method of claim 40, wherein the biological constructs are labeled.

45. The method of claim 44, wherein the label is selected from the group consisting of colormetric dye, fluorescent dye, chemiluminescent dye, quantum dots, magnetic particle label, and fluorescent particle label.

46. The method of claim 40, wherein the biological constructs are detected by a method selected from the group consisting of gravimetric sensing and phase contrast microscopy.

47. The method of claim 40, wherein the ALR is selected from the group comprising polyclonal antibodies, monoclonal antibodies, single chain antibodies (scFv), Fab fragments, F(ab′)₂ fragments, Fv fragments, small organic molecules, and polypeptides.

48. The method of claim 40, wherein the biological construct is selected from the group consisting of cells, bacteria, viruses, fungi, protozoa, and parasites.

49. The method of claim 40, wherein the different biological constructs comprise different strains of the same biological construct.

50. The method of claim 40, wherein the solid support is selected from the group consisting of glass, nitrocellulose, nylon, silicon wafers, microfabricated sensors, polystyrene, and polyvinyl chloride.

51. The method of claim 40, wherein the sample is selected from the group consisting of a biological sample, a water sample, an environmental sample, and a food sample.

52. A method of providing information concerning biological construct ALR binding patterns to a first computer device through a second computer device, comprising the steps of:
(a) receiving at least one ALR binding pattern from the first computer device;
(b) comparing the at least one ALR binding pattern received from the first computer device to records in an ALR binding pattern information database; and
(c) compiling a list of matching biological construct information from the database records matching the at least one ALR binding pattern received from the first computer device.

53. The method of claim 52, wherein the second computer device is selected from the group of devices consisting of a web server, a stand alone computer, and a personal digital assistant.

54. The method of claim 52, wherein the first computer device and the second computer device are connected by a network.

55. A computer readable medium having stored therein instructions for causing a central processing unit to execute the method of claim 52.

56. The method of claim 52, further comprising visually displaying the list on a display device.

57. A computer readable medium having stored therein instructions for visually displaying the list of claim 52.

58. The method of claim 52, wherein the step of comparing comprises:
(a) obtaining a binding pattern from the first computer device;
(b) obtaining a binding pattern record from the database;
(c) comparing the binding patterns of steps (a) and (b);
(d) repeating step (c) until a match is made.

59. The method of claim 58, wherein step (c) is repeated until all binding pattern records in the database are compared to the binding pattern from the first computer device.

60. A method of providing information concerning biological construct or receptor ALR binding patterns and biological construct or receptor quantity to a first computer device through a second computer device, comprising the steps of:
(a) receiving at least one ALR binding pattern comprising binding intensities from a first computer device;
(b) comparing the at least one ALR binding pattern received from a first computer device to records in an ALR binding pattern and binding intensity information database; and
(c) compiling a list of records matching the at least one ALR binding pattern comprising binding intensities received from a first computer device.

61. The method of claim 60, wherein the second computer device is selected from the group of devices consisting of a web server, a stand alone computer, and a personal digital assistant.

62. The method of claim 60, wherein the first computer device and the second computer device are connected by a network.

63. A computer readable medium having stored therein instructions for causing a central processing unit to execute the method of claim 60.

64. The method of claim 60, further comprising visually displaying the list on a display device.

65. A computer readable medium having stored therein instructions for visually displaying the list of claim 60.

66. The method of claim 60, wherein the step of comparing comprises:
(a) obtaining a binding pattern comprising binding intensities from the first computer device;
(b) obtaining a binding pattern record comprising binding intensities from the database;
(c) comparing the binding patterns and intensities of steps (a) and (b);
(d) repeating step (c) until a match is made.

67. The method of claim 66, wherein step (c) is repeated until all records of binding patterns comprising binding intensities in the database are compared to the binding pattern comprising binding intensities from the first computer device.

68. A system allowing users to obtain information on the identity or quantity of biological constructs or receptors in a sample from a directory available via a computer comprising in combination:
(a) a second computer device in communication with a first computer device to allow users to enter selection criteria for retrieving records of biological construct or receptor ALR binding patterns and binding intensities; and
(b) a database comprising records of biological construct or receptor ALR binding patterns and binding intensities,
(c) wherein the second computer device produces a list of matching ALR binding pattern and binding intensity records from the database that match the selection criteria and displays the matching pattern and binding intensity records on the list in an order determined by each matching biological construct's or receptor's similarity to the selection criteria.

69. The system of claim 68, wherein the first computer device and the second computer device are connected by a network.

70. The system of claim 68, wherein the selection criteria comprises an ALR binding pattern provided by the first computer device.

71. The system of claim 68, wherein the selection criteria comprises an ALR binding intensity provided by the first computer device.

72. The system of claim 68, wherein the selection criteria comprises an ALR binding pattern and an ALR binding intensity provided by the first computer device.

73. The system of claim 68, wherein the ALR binding pattern and intensity provided by the first computer device is determined by applying a sample suspected of containing biological constructs or receptors to a solid support comprising ALRs specific for at least one type of biological construct or receptor, wherein at least about 10 different ALRs capable of specifically binding to each type of biological construct or receptor are bound to the solid support in predefined locations.

74. The system of claim 68, wherein the network device is selected from the group of devices consisting of a web server, a stand alone computer, and a personal digital assistant.

75. The system of claim 68, further comprising visual display of the list on a display device.

76. A computer readable medium having stored therein instructions for visually displaying the list of claim 68.
